(19) United States
(12) Patent Application Publication
(10) Pub. No.: US 2008/0228249 A1
(43) Pub. Date: Sep. 18, 2008

(54) MEDICAL DEVICE WITH THERMAL MANAGEMENT OF THE DEVICE-TISSUE INTERFACE

(76) Inventor: James C. Barnitz, Schwenksville, PA (US)

Correspondence Address: FOX ROTHSCHILD LLP;PRINCETON PIKE CORPORATE CENTER, 2000 Market Street, Tenth Floor, Philadelphia, PA, 19103, US

(21) Appl. No.: 11/685,053

(22) Filed: Mar. 12, 2007

(51) Int. Cl. A61F 7/12

(52) U.S. Cl. 607/113

(57) ABSTRACT

A medical device includes a heat-generating region and a thermally conductive body thermally and mechanically coupled to the heat-generating region. The conductive body encloses a cavity that is filled with thermal ballast. The cavity ideally extends along at least 50% of the length of the conductive body. The thermal ballast may be a phase-change material.

### BACKGROUND OF THE INVENTION

A number of medical devices currently exist that cause tissue heating at the tissue-device interface, which is typically the distal end of the device. This tissue heating may be intentionally induced, such as in cauterizing or coagulating devices, or as a by-product of energy imparted to the device for other purposes, as with ultrasonic scalpels.

Although heating may or may not be the desired approach for affecting or examining the tissue, a number of complications associated with the interaction between medical devices and heated tissue may arise. For example, as the tissue temperature at the device-tissue interface begins to approach 100° C., the aqueous contents of the tissue cells begin to evaporate, and the tissue becomes desiccated. This desiccation of the tissue changes the electrical and other material properties of the tissue, and may impede treatment. The desiccated tissue may also adhere to the device, causing tearing of the tissue and the need to periodically stop and clean the adhered tissue from the device. These adverse effects can be even more severe if the tissue at the interface exceeds 100° C. and the solid contents of the tissue begin to char.

Excessive temperatures at the device-tissue interface can thus cause complications, and cooling of the device-tissue interface may be required. However, if the medical device requires an elevated temperature to achieve the desired tissue effect, overcooling can prevent proper treatment. As an example, coagulation requires a minimum temperature of approximately 60° C. to solidify proteins and stop bleeding; if the cooling device has an excessively high heat transfer rate, the tissue at the interface will not achieve the treatment temperature and coagulation will not occur.

Methods and related systems for cooling a device-tissue interface of a medical device are known. However, these systems tend to be either complicated, bulky or both, particularly for minimally invasive devices. Accordingly, there is an immediate need to provide for the cooling of a tissue-device interface that is relatively easy to implement and that is effective across a broad range of devices and circumstances.

### SUMMARY OF THE INVENTION

Methods and related devices are disclosed that provide for the removal of heat from a device-tissue interface of a medical device. In one aspect, a medical device is provided that includes either a heat-generating end that serves as the tissue-device interface or a tissue-device interface that is heated by conduction from tissue at an elevated temperature. In some embodiments, the heat-generating end may deliberately induce heating as part of a treatment. In other embodiments, the heat-generating end creates heat as a by-product of another energetic activity performed for treatment purposes. A thermally conductive body is both mechanically and thermally attached to the device-tissue interface. The thermally conductive body provides a radiative surface that rejects heat into the ambient environment. In some embodiments the thermally conductive body is integrally formed with the device-tissue interface. Thermal connection of the device-tissue interface to the thermally conductive body provides a first thermal pathway that allows heat from the device-tissue interface to conduct to the radiative surface, and thence be rejected into the ambient environment. The thermally conductive body surrounds a cavity that is filled with a thermal ballast. A second thermal pathway is thus provided that permits heat from the device-tissue interface to move into the thermal ballast. A third thermal pathway permits heat contained within the thermal ballast to migrate into the radiative surface, and thus be rejected into the ambient environment. In some embodiments the thermal ballast is provided by a sensible material with a relatively high specific heat capacity. The sensible material preferably has a specific heat capacity of at least 4170 joules/° K·kg. In other embodiments the thermal ballast is provided by a phase-change material. In certain preferred embodiments the phase-change material undergoes a phase change near the desired operating temperature of the device-tissue interface.

Another aspect provides a method for removing heat from a device-tissue interface in a medical device. The method includes providing a first thermal pathway from the device-tissue interface to a radiative surface on the medical device, providing a second thermal pathway from the device-tissue interface to a thermal ballast adapted to store thermal energy, and providing a third thermal pathway from the thermal ballast to the radiative surface. The first thermal pathway is a conductive pathway. The second and third thermal pathways may be conductive pathways, or combined conductive and convective pathways. Certain preferred embodiments of the method further include enclosing the thermal ballast with the radiative surface.

### BRIEF DESCRIPTION OF THE DRAWINGS

FIG. 1 is a side view of an embodiment medical device.

FIG. 2 shows the removing of a plug in the medical device depicted in FIG. 1.

FIG. 3 is a side view of another embodiment medical device.

### DETAILED DESCRIPTION

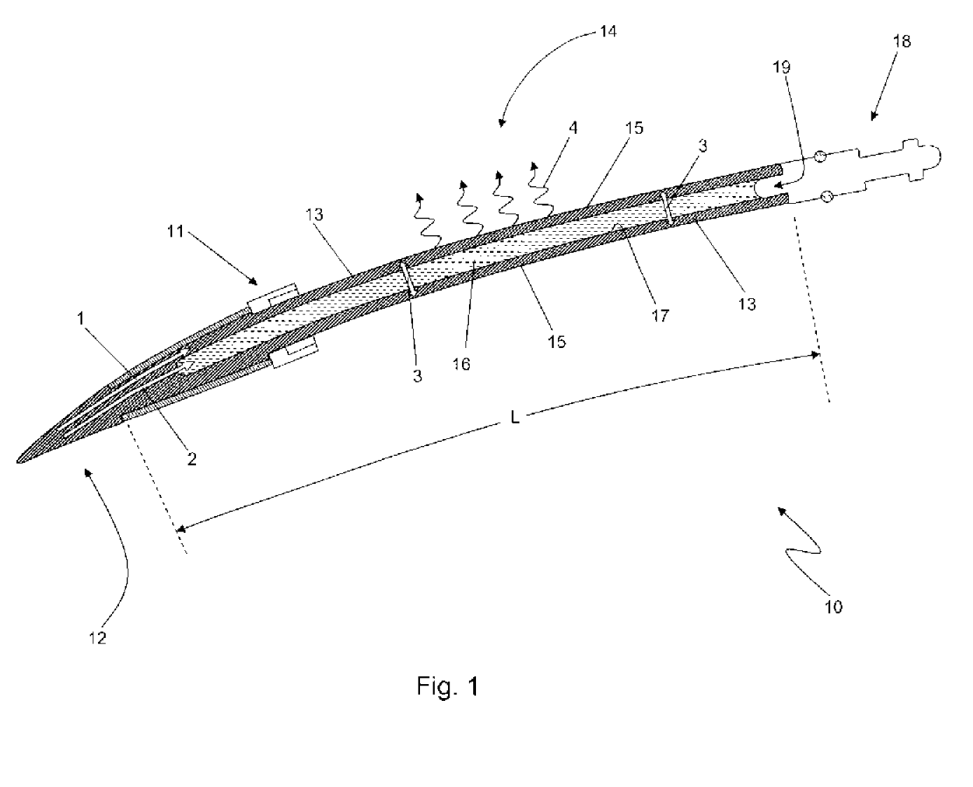

FIG. 1 is a side view of a medical device 10 that employs an embodiment cooling method. Although the medical device 10 shown in FIG. 1 is a forceps, it will be appreciated that any medical device, such as cutting forceps, scissors, and scalpel may benefit from the cooling systems and methods described in the following.

The device 10 includes a device-tissue interface 12, which is the region in which the device 10 contacts the tissue of a patient to perform a medical procedure. The specific type of procedure performed will, of course, depend upon the type classification of the medical device 10. For example, the device-tissue interface 12 may perform cutting, as with an ultrasonic scalpel; cauterizing; coagulation, or any other of a myriad of processes that generally involve the application of energy to tissue. The device-tissue interface 12 is at an elevated temperature due to energy that is applied in or near the interface 12. The heating of the device-tissue interface 12 may be deliberate, as is the case with cauterizing and coagulation procedures, or may be a by-product of the desired operation, as is the case with ultrasonic scalpels, in which the mechanical energy of the ultrasonic vibration of the interface 12 causes the interface 12 to generate heat.

Various embodiments take advantage of the fact that the interface 12 tends to operate only intermittently. That is, a user of the device 10, such as a surgeon, does not continuously activate the device 10, but rather tends to use the device 10 at intervals. When activated, the device-tissue interface 12 generates or accepts thermal energy, and hence the temperature of the device-tissue interface 12 will begin to rise unless this thermal energy is removed. However, once the surgeon turns the device 10 off, the device-tissue interface 12 is no longer heated. During these lulls, heat generated during previous cycles of use may be rejected to the ambient environment. The ambient environment is typically the air within the operating room or locale in which the device 10 is used, but may be, for example, the blood of the patient, depending upon the type classification of the device 10. Certain types of catheters, for example, may reject heat by dumping the excess heat into the bloodstream of the patient. Broadly speaking, the device 10 employs a cooling method that both rejects to ambient as much heat as possible while the device 10 is in use and stores the remaining heat in a thermal ballast. Then, during quiescent periods, the stored thermal energy is rejected to ambient.

For any device to reject heat to ambient a radiating surface is required. The amount of heat rejected per unit time will primarily depend upon the area of the radiating surface in contact with the ambient environment, and the temperature of this area, although other thermodynamic factors, such as color, also play a role. Although the term “radiating” formally means the rejection of heat through the radiation of electromagnetic radiation, the term is more broadly understood to mean the rejection of heat to ambient through both convection and radiation. The “radiator” in a car, for example, primarily rejects heat through convection, not radiation. In the following, then, a radiating surface should be understood to reject heat through convection, radiation or both of thermal energy to an ambient environment.

A thermally conductive body 14 of the device 10 provides a radiative surface 15 that is used to reject heat to ambient. The radiative surface 15 is preferably at least 25% of the external surface area of the body 14, and even more preferentially at least 50% of the body 14 external surface area. The radiative surface 15 is directly coupled to the ambient environment so as to reject heat into the ambient environment.

The thermally conductive body 14 ideally has a thermal conductivity of at least 200 W/m·° K. Materials suitable for the thermally conductive body 14 include gold, silver, copper, and aluminum. Although not indicated in FIG. 1, the thermally conductive body 14 may be a laminated or non-monolithic device; however, each of the individual components or layers within the body 14 should be sufficiently thermally conductive as to provide the entire structure 14 with a thermal conductivity of at least 150 W/m·° K. The thermally conductive body 14 is both mechanically and thermally connected to the device-tissue interface 12. In certain embodiments, as shown in FIG. 1, the thermally conductive body 14 may be integrally formed with the device-tissue interface 12. In other embodiments, the device-tissue interface 12 may be mechanically coupled to the body 14 by any suitable means, including soldering, welding or other like permanent connections, or may be removably attached to the body 14, such as with a threaded connection, a locking-type connection or any other such suitable connection. In preferred embodiments, the device-tissue interface 12 is also made from a thermally conductive material, having a thermal conductivity of at least 200 W/m·° K. Suitable materials for the device-tissue interface 12 include gold, silver, copper, and aluminum. The device-tissue interface 12 may also have a laminate or non-monolithic structure. Heat present in the device-tissue interface 12 may flow by way of conduction through the device-tissue interface 12 and into the sidewalls 13 of the thermally conductive body 14, as shown by arrow 1. From the sidewalls 13 the heat may reach, through conduction, the radiative surface 15, and thence be rejected into the ambient environment, as shown by arrows 4. Arrow 1 thus depicts a first thermal pathway along which heat present at the device-tissue interface 12 may travel for rejection out to ambient.

The amount of heat per unit time, i.e., the radiative power, of the radiative surface 15 is a limited quantity, which depends on several factors, such as the surface area of the radiative surface 15, the material properties of the radiative surface 15 and the temperature of the radiative surface 15. However, while operating, the device-tissue interface 12 may generate more heat per unit time, i.e., have a higher thermal power rating, than the radiative surface 15 can handle. That is, the heating power of the device-tissue interface 12 may exceed the cooling power of the radiative surface 15. The temperatures of the device-tissue interface 12 and the radiative surface 15 will both begin to rise if this excess heat cannot be dumped somewhere. The device 10 provides for temporary storage of this excess heat by making use of a thermal ballast 16.

The thermally conductive body 14 comprises a cavity 17. The cavity 17 may be, for example, a lumen that is surrounded by the sidewalls 13 of the thermally conductive body 14. In certain embodiments, the cavity 17 extends along at least 25% of the length of greatest extent L of the thermally conductive body 14. Disposed within and filling the cavity 17 is the thermal ballast 16. The thermal ballast 16 is ideally a material capable of storing relatively large amounts of heat without experiencing significant changes in temperature while the device-tissue interface 12 is operating at or near a desired process temperature. This may be provided, for example, by a sensible material or by a phase-change material. To provide for maximum heat-storage capabilities within the cavity 17, as much thermal ballast 16 as possible should be disposed within the cavity 17, while providing sufficient space to accommodate for thermal expansion and other factors. The thermal ballast 16 remains sealed within the cavity 17 during use and operation of the medical device 10. That is, while the practitioner is using the device 10, little and ideally none of the thermal ballast 16 escapes the confines of the cavity 17, which is preferably sealed along the entire length L of the thermally conductive body 14. Nevertheless, for safety reasons it may be desirable to select non-toxic materials for the thermal ballast 16.

A sensible material is one which remains within the same phase, typically solid or liquid, across all reasonable operating temperatures of the device-tissue interface 12. If a sensible material is used for the thermal ballast 16, then the sensible material 16 is preferably selected from materials having a high specific heat capacity, such as in excess of 4170 joules/° K kg. Examples of suitable sensible materials include water and saline.

If a phase-change material is used for the thermal ballast 16, then the type of phase-change material used may depend upon the desired operating temperature of the device-tissue interface 12. The phase change material 16 preferably undergoes a phase transition, such as from solid to liquid, at a temperature that is near the desired operating temperature of the device-tissue interface 12; more preferably, the phase transition should occur at or just below the desired operating temperature of the device-tissue interface 12. For example, if the desired operating temperature of the device-tissue interface 12 is 60° C., then it may be desirable to pick as the thermal ballast 16 a material that undergoes a phase change near 55° C. to 60° C. Examples of suitable phase-change materials include organic paraffin, organic non-paraffin and inorganic salt hydrate.

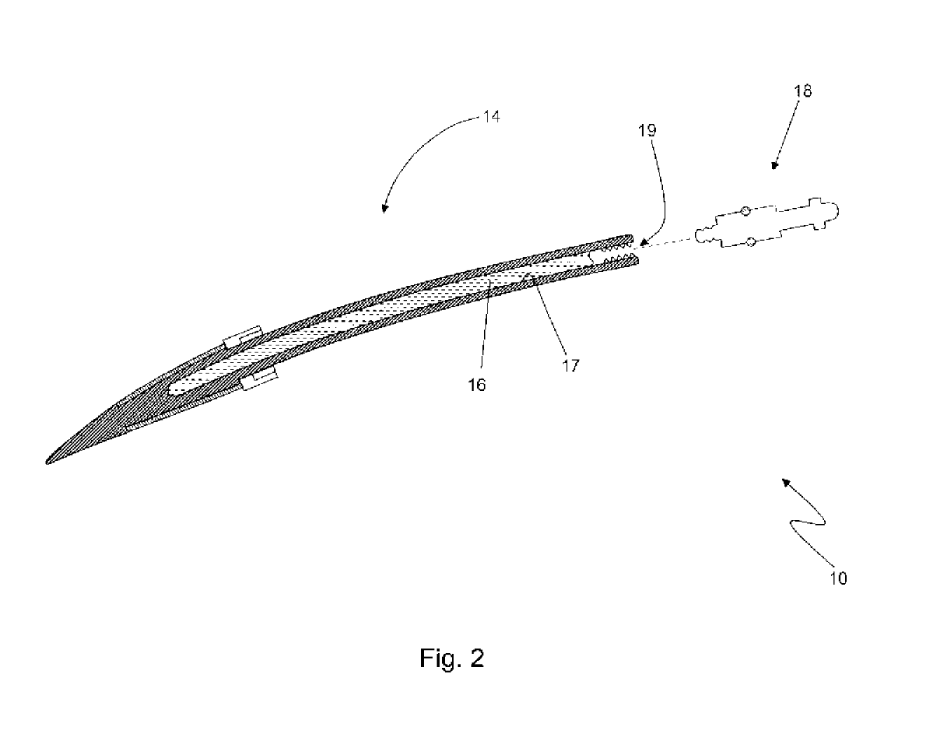

Although the thermal ballast 16 should remain sealed within the cavity 17 while the device 10 is in use, it may be desirable to enable the practitioner to replace the thermal ballast 16. This may advantageously provide for greater flexibility in setting the desired operating temperature of the device-tissue interface 12. For example, a thermal ballast 16 that undergoes a phase-change at 80° C. may be swapped out for another thermal ballast 16 that undergoes a phase-change at 60° C. so as to accommodate different process temperatures for the particular procedures that are to be performed by the medical device 10. With further reference to FIG. 2, the device 10 may be provided a removable cap or plug 18 that removably seals an opening 19 in the thermally conductive body 14. The opening 19 provides access to the cavity 17, and hence allows the thermal ballast 16 to be replaced. Any suitable mechanism may be used to provide the removable seal between the plug 18 and the opening 19, such as a threaded connection, a friction fitting, a Luer-lock, etc., and which may be used in combination with zero or more gaskets disposed between the plug 18 and the body 14 to provide a tight seal.

As the thermal ballast 16 is disposed within the cavity 17 of the thermally conductive body 14, and as the thermally conductive body 14 is thermally connected to the device-tissue interface 12, a second thermal pathway exists, indicated by arrow 2, along which heat present at the device-tissue interface 12 may travel for eventual rejection out to the ambient environment. Heat may travel, by way of conduction, through the device-tissue interface 12 and into the thermal ballast 16. The heat may then flow within the thermal ballast 16 either through conduction or convection, depending upon the phase of the thermal ballast 16. The second thermal pathway 2 may thus be a conduction-only pathway, or a combined conduction-convection pathway. With specific reference to a phase change material, a significant amount of the heat flowing along the pathway 2 is absorbed and used to change the phase of the thermal ballast 16 without actually raising the temperature of the thermal ballast 16. On the other hand, with sensible materials, a great deal of heat must travel along the second thermal pathway 2 to significantly raise the temperature of the thermal ballast 16. In both cases, the thermal ballast 16 acts as temporary buffer for the thermal energy traveling along the second thermal pathway 2. Heat that is unable to travel along the first thermal pathway 1 due to the limited radiating power of the radiative surface 15 travels along the second thermal pathway 2 for temporary storage in the thermal ballast 16. Of course, second thermal pathway 2 may also include heat traveling from device-tissue interface 12, through the sidewalls 13 and then into the thermal ballast 16.

Because the sidewalls 13 of the body 14 are thermally conductive and in direct contact with the thermal ballast 16, a third thermal pathway 3 exists that enables heat to flow from the thermal ballast 16 out to the radiative surface 15 for rejection into the ambient environment. When the amount of heat traveling along the first thermal pathway 1 drops below the radiating capabilities of the radiative surface 15, as happens when the device 10 is not operating and thus no longer actively generating heat, the power difference can be made up for by heat stored in the thermal ballast 16 traveling along the third thermal pathway 3 to the radiative surface 15. The stored thermal energy within the thermal ballast 16 is thus rejected during quiescent or non-operational periods of the device 10. On the other hand, when the device 10 is operational and generating heat at the device-tissue interface 12 at a rate that exceeds the rejecting capacity of the radiative surface 15, the excess heat is stored in the thermal ballast 16. Thermal ballast 16 thus provides for a time-averaged rejection of the heat pulled from the device-tissue interface 12.

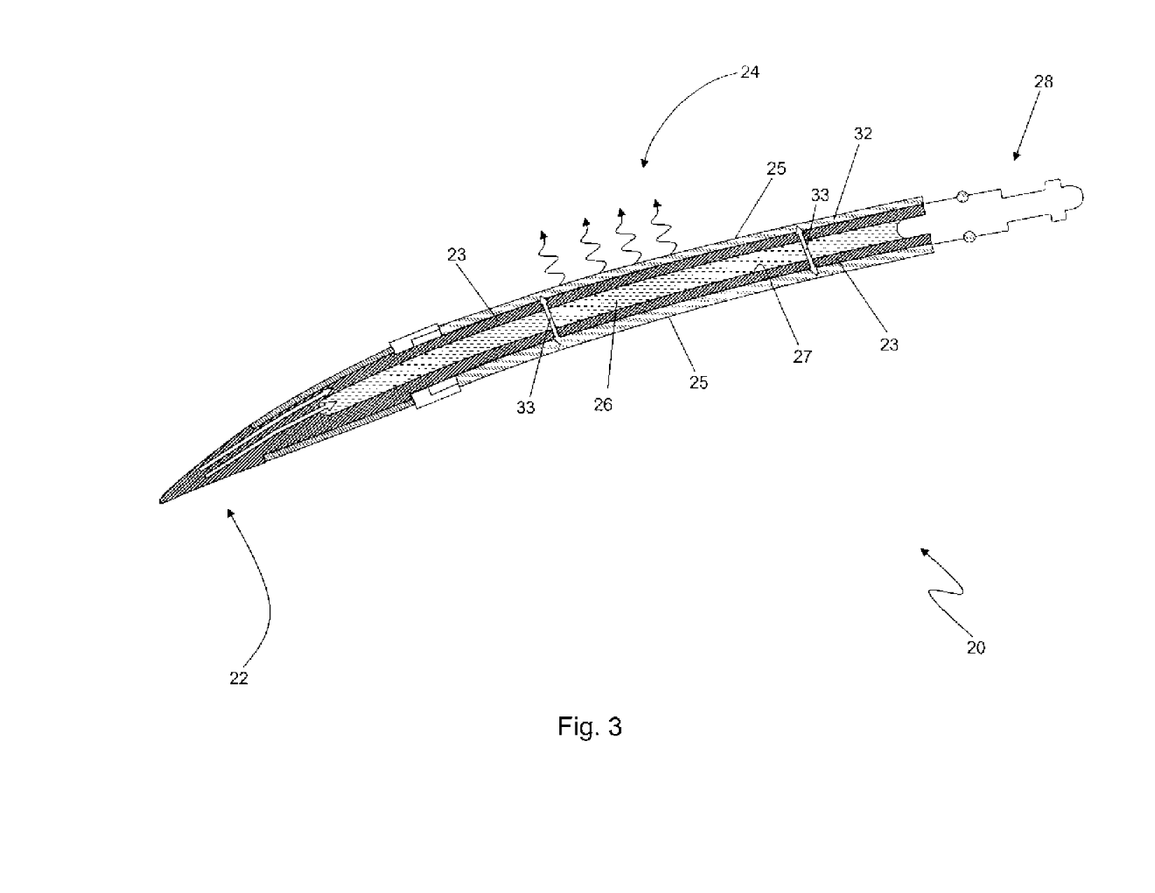

In certain embodiments electrical energy may flow along a portion of the body to provide energy to the device-tissue interface. In such embodiments it may be desirable to provide an electrically insulative material that covers the body so as to protect the practitioner from shock-related hazards. As shown in FIG. 3, a second embodiment medical device 20 includes a device-tissue interface 22 mechanically and thermally coupled to a thermally conductive body 24. The thermally conductive body 24 provides a radiative surface 25, and surrounds a cavity 27 that is filled with a thermal ballast 26. The thermally conductive body 24 comprises internal sidewalls 23 formed from a material that is both electrically and thermally conductive, and an external sheath 32 that is thermally conductive but electrically insulative. The external sheath 32 preferably has a thermal conductivity of at least 0.20 W/m·° K. As shown by arrows 33, thermal energy stored in the thermal ballast 26 may flow, by way of conduction, through the internal sidewalls 23, through the sheath 32 and to the radiative surface 25 for rejection to ambient. Suitable materials for the external sheath 32 include nylon, fluorinated ethylene-propylene copolymer and polyvinylidene fluoride. Electrical energy may flow along the internal sidewalls 23 to the device-tissue interface 22, or to an electrical component disposed within or near the device-tissue interface 22, while the sheath 32 protects the practitioner from electrical shock. Plug 28 may be used as an electrical attachment point for connecting the device 20 to a cord or wire that provides electricity from a power source.

In certain embodiments the operation of the devices occurs at variable power levels. By way of example only the radiative surface is designed to handle the maximum power setting, such that the device would be over cooled when it is operating at lower power settings. In other embodiments of the invention the radiative surface may be designed for the lower power setting such that when operated at higher power setting the excess heat could be stored and released when the device is off. This approach would work well with a phase change material since the melt temperature could be selected to be higher than the minimum desired operating temperature.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the following claims.

What is claimed is:

1. A medical device comprising:
a device-tissue interface;
a thermally conductive body thermally coupled to the ambient environment and directly thermally and physically coupled to the device-tissue interface, the thermally conductive body comprising a cavity; and
thermal ballast disposed within and substantially filling the cavity.

2. The medical device of claim 1 wherein the cavity is sealed.

3. The medical device of claim 2 wherein the cavity comprises a sealable opening.

4. The medical device of claim 1 wherein the thermal ballast is selected from a set consisting of materials that undergo a phase change at a temperature near a desired operating temperature of the heat-generating region.

5. The medical device of claim 1 wherein the thermal ballast has a specific heat capacity of at least 4170 joules/° K kg.

6. The medical device of claim 1 wherein at least a portion of the thermally conductive body is integrally formed with the device-tissue interface.

7. The medical device of claim 1 wherein the cavity extends along at least 25% of a longitudinal length of the thermally conductive body.

8. The medical device of claim 1 wherein the thermally conductive body thermally couples to the ambient environment through a radiative surface that comprises at least 25% of the external surface area of the thermally conductive body.

9. A method for removing heat from a device-tissue interface in a medical device, the method comprising:
providing a first thermal pathway from the device-tissue interface to a radiative surface on the medical device;
providing a second thermal pathway from the device-tissue interface to a thermal ballast adapted to store thermal energy; and
providing a third thermal pathway from the thermal ballast to the radiative surface.

10. The method of claim 9 wherein the first thermal pathway is a conductive pathway.

11. The method of claim 9 wherein the second thermal pathway is a conductive pathway, or a combined conductive and convective pathway.

12. The method of claim 9 wherein the third thermal pathway is a conductive pathway, or a combined conductive and convective pathway.

13. The method of claim 9 further comprising enclosing the thermal ballast with the radiative surface.
